(19) United States
(12) Patent Application Publication
(10) Pub. No.: US 2021/0137513 A1
(43) Pub. Date: May 13, 2021

(54) HEALICOIL KNOTLESS OUTER DRIVER RETENTION FEATURE

(71) Applicants: Smith & Nephew, Inc., Memphis, TN (US); Smith & Nephew Orthopaedics AG, Baar (CH); Smith & Nephew Pte. Limited, Singapore (SG)

(72) Inventors: Jon-Paul Rogers, North Smithfield, RI (US); Mark Edwin Housman, North Attleborough, MA (US); Peter James Cashmore, Durham, NH (US)

(73) Assignee: Smith & Nephew, Inc., Memphis, TN (US)

(21) Appl. No.: 16/679,504

(22) Filed: Nov. 11, 2019

(51) Int. Cl. A61B 17/04

(52) U.S. Cl. CPC A61B 17/0401; A61B 2017/0456; A61B 2017/0427; A61B 2017/044

(57) ABSTRACT

A system for tissue repair includes an anchor body having a proximal end, a distal end, and a plurality of turns of a screw thread extending between the proximal and distal ends. The plurality of turns of the screw thread define an internal volume communicating with a region exterior to the anchor body through a spacing defined between the plurality of turns of the screw thread, and at least one rib disposed within the internal volume connected to at least two of the plurality of turns of the screw thread. The system includes an anchor driver comprising an elongated shaft extending between a proximal end and a distal end. A distal portion of the shaft includes an open slot defined by at least two distally-extending prongs. The at least one rib of the anchor body is engageable with the open slot of the shaft of the anchor driver.

### FIELD OF THE INVENTION

The described technology relates generally to tissue repair, and more specifically, to an anchor for securing tissue to bone.

### BACKGROUND OF THE INVENTION

Arthroscopic procedures often require soft tissue to be reattached to bone. To achieve this, anchors are placed in the bone and sutures attached to the anchor are passed through the tissue to securely retain the tissue in place. When making a repair of soft tissue to bone, it is advantageous to have as large an area of contact between the bone and tissue as possible. Anchor points spaced from one another in rows result in a repair having a broader area of contact.

### BRIEF SUMMARY OF THE INVENTION

A procedure, and components for use in such procedure, that securely attaches tissue to bone using a plurality of attachment points over a large area of contact is needed. Such procedure must be able to be done in a quick and efficient manner with a minimum of recovery time for the patient.

In one aspect, the present disclosure relates to a system for tissue repair. The system can include an anchor body having a proximal end, a distal end, and a plurality of turns of a screw thread extending between the proximal and distal ends. The plurality of turns of the screw thread defines an internal volume communicating with a region exterior to the anchor body through a spacing defined between the plurality of turns of the screw thread. Further, the anchor body includes at least one rib disposed within the internal volume connected to at least two of the plurality of turns of the screw thread. The system can include an anchor driver comprising an elongated shaft extending between a proximal end and a distal end, a distal portion of the shaft including an open slot defined by at least two distally-extending prongs. The at least one rib of the anchor body can be engageable with the open slot of the shaft of the anchor driver; and an inner surface of at least one of the at least two prongs defines a protrusion for forming an interference fit with the at least one rib.

In some embodiments, the anchor body includes a plurality of ribs disposed within the internal volume. The anchor driver can include a plurality of open slots, each open slot can be configured to receive the respective rib of the plurality of ribs. The inner surface of at least one of the at least two prongs can have a distal end and a proximal end. In some embodiments, the protrusion is positioned on the distal end of the inner surface of at least one of the at least two prongs. In other embodiments, the protrusion is positioned on the proximal end of the inner surface of at least one of the at least two prongs.

In some embodiments, each of the plurality of open slots is defined by two distally-extending prongs. In some embodiments, an inner surface of at least one of the two prongs of each open slot defines a protrusion for forming an interference fit with the respective rib of the plurality of ribs.

In one aspect, the present disclosure relates to a method of tissue repair. The method can include driving an anchor of an anchor system into a bone hole. The anchor system can include an anchor body having a proximal end, a distal end, and a plurality of turns of a screw thread extending between the proximal and distal ends. The plurality of turns of the screw thread can define an internal volume to communicate with a region exterior to the anchor body through a space defined between the plurality of turns of the screw thread. At least one rib can be disposed within the internal volume connected to at least two of the plurality of turns of the screw thread. Further, the method can include an anchor driver comprising an elongated shaft extending between a proximal end and a distal end, a distal portion of the shaft including an open slot defined by at least two distally-extending prongs. The at least one rib of the anchor body can be engageable with the open slot of the shaft of the anchor driver and an inner surface of at least one of the at least two prongs can define a protrusion for forming an interference fit with the at least one rib. The method can further include advancing the anchor body into the bone using the anchor driver.

### BRIEF DESCRIPTION OF THE DRAWINGS

Various aspects of at least one embodiment of the present disclosure are discussed below with reference to the accompanying figures. It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. For purposes of clarity, not every component may be labeled in every drawing. The figures are provided for the purposes of illustration and explanation and are not intended as a definition of the limits of the invention.

FIG. 1 illustrates an system for tissue repair, according to certain embodiments; and

FIG. 2 illustrates a system for tissue repair, according to certain embodiments.

### DETAILED DESCRIPTION

In the following detailed description, numerous specific details are set forth in order to provide a thorough understanding of the embodiments of the present disclosure. It will be understood by those of ordinary skill in the art that these embodiments may be practiced without some of these specific details. In other instances, well-known methods, procedures, components and structures may not have been described in detail so as not to obscure the described embodiments.

Prior to describing at least one embodiment in detail, it is to be understood that the claims are not limited in their application to the details of construction and the arrangement of the components set forth in the following description or illustrated in the drawings. Also, it is to be understood that the phraseology and terminology employed herein are for the purpose of description only and should not be regarded as limiting.

Described herein is a rotator cuff lateral anchor which has an open healicoil proximal implant (e.g., anchor body) with internal ribs for mating with grooves or slots on an inserter driver when the inserter (anchor) driver is inserted into the internal volume of the proximal implant. During a surgical procedure, the proximal implant is required to stay on the inserter driver while entering an arthroscopic portal (i.e., a cannula or a percutaneous incision), as well as during manipulation and insertion of the proximal implant into bone. Manipulation during insertion may include mallet/striking forces on a proximal portion of the inserter driver. The mallet forces can dislodge the implant from the inserter driver. However, the mallet forces are needed to insert the implant. If the proximal implant migrates off of the inserter, it may compromise the procedure, for example, by occluding visualization or anchor fracture. Alternatively, if the proximal implant fully falls off the inserter, it becomes a “loose body” in the joint space, requiring retrieval.

The present disclosure describes an implant and an inserter instrument with a protrusion to aid in keeping the implant on the inserter. The implant can be secured to an inserter middle shaft described in U.S. Pat. No. 9,526,488, incorporated by reference herein. Accordingly, the present disclosure describes a device in which the inserter driver incorporates a finely controlled protrusion/press to aid in keeping the proximal implant on the inserter. For example, the protrusion/press may be disposed on an inner surface of one of the distal forks of the inserter driver for forming an interference fit with the mating internal rib of the proximal implant. This single point of contact allows for a controlled fit between the proximal implant and the inserter and assures that the interference fit is neither too tight nor too loose. This also allows for a greater tolerance window, as the interference fit occurs only at specific points along the inserter.

The present disclosure provides a system for tissue repair. FIG. 1 illustrates a tissue repair system 100 being disassembled, according to certain embodiments. The tissue repair system 100 includes an anchor body 102 having a proximal end 104, a distal end 106, and a plurality of turns of a screw thread 108 extending between the proximal end 104 and the distal end 106. The plurality of turns of the screw thread 108 defines an internal volume 110 which communicates with a region 112 exterior to the anchor body 102 through a spacing 114. The spacing 114 is defined between the plurality of turns of the screw thread 108. The anchor body 102 includes a rib 116 disposed within the internal volume 110. The rib 116 is connected to at least two of the plurality of turns of the screw thread 108. In some embodiments, the anchor body 102 includes two ribs 116 which are connected to at least two of the plurality of turns of the screw thread 108. In some embodiments, the ribs 116 of the anchor body 102 are connected to more than two of the plurality of turns of the screw thread 108.

In other embodiments, the anchor body 102 includes a plurality of ribs 116 being disposed within the internal volume 110.

As FIG. 1 illustrates, the tissue repair system 100 includes an anchor driver 120. The anchor driver 120 includes an elongated shaft 122 extending between a proximal end 124 and a distal end 126. Further, the anchor driver 120 includes a distal portion 128. The distal portion 128 of the elongated shaft 122 includes an open slot 130 defined by at least two distally-extending prongs 132, 134. In some embodiments, the rib 116 of the anchor body 102 is engaged with the open slot 130 of the shaft 122 of the anchor driver 120. In other embodiments, the anchor driver 120 includes a plurality of open slots 130. Each open slot 130 is configured to receive the respective rib 116 of the plurality of ribs 116. In some embodiments, each open slot 130 of the plurality of the open slots is defined by two distally-extending prongs 132, 134.

In some embodiments, the prongs 132, 134 have an inner surface 136. The inner surface 136 of at least one of the at least two prongs 132, 134 includes a protrusion 140. The protrusion 140 forms an interference fit with the at least one rib 116 of the anchor body 102. The protrusion 140 can have any geometrical shape. In some embodiments, the protrusion 140 is a square.

The inner surface 136 of at least one of the at least two prongs 132, 134 have a distal end 135 and a proximal end 137. The protrusion 140 can be located on any place on the inner surface 136 of at least one of the at least two prongs 132, 134. In some embodiments, the protrusion 140 is positioned on the distal end 135 of the inner surface 136 of prong 132. In other embodiments, the protrusion 140 is positioned on the proximal end 137 of the inner surface 136 of prong 132.

As mentioned above, each open slot 130 of the plurality of the open slots is defined by two distally-extending prongs 132, 134. In some embodiments, one of the two distally-extending prongs 132, 134, includes a protrusion on the inner surface 136. In some embodiments, both prongs of the two distally-extending prongs 132, 134 have protrusions 140 which are located on the inner surface 136, 136′. Accordingly, in this embodiment, all open slots 130 include protrusions 140 for forming interface fits with the respective rib 116 of the plurality of ribs 116.

Some of the advantages of the protrusion 140 are to provide a single point contact which allows for a controlled fit between the proximal implant 102 and the anchor driver 120. Accordingly, it provides assurance that the interference fit is neither too tight nor too loose. The single point contact allows for a greater tolerance window as the interface is only at specific points.

FIG. 2 illustrates a system for tissue repair 200, according to certain embodiments. The tissue repair system 200 includes an anchor body 202 having a proximal end 204, a distal end 206, and a plurality of turns of a screw thread 208 extending between the proximal end 204 and the distal end 206. The plurality of turns of the screw thread 208 defines an internal volume 210 which communicates with a region 212 exterior to the anchor body 202 through a spacing 214. The spacing 214 is defined between the plurality of turns of the screw thread 208. The anchor body 202 includes ribs 216 disposed within the internal volume 210. The ribs 216 are connected to at least two of the plurality of turns of the screw thread 208. In some embodiments, the anchor body 202 includes four ribs 216 which are connected to at least two of the plurality of turns of the screw thread 208. In some embodiments, the ribs 216 of the anchor body 202 are connected to more than two of the plurality of turns of the screw thread 208.

As FIG. 2 illustrates, the tissue repair system 200 includes an anchor driver 220. The anchor driver 220 includes an elongated shaft 222 extending between a proximal end 224 and a distal end 226. Further, the anchor driver 220 includes a distal portion 228. The distal portion 228 of the elongated shaft 222 includes a plurality of open slots 230. Each open slot 230 of the plurality of open slots 230 is defined by at least two distally-extending prongs e.g., 232, 234. The ribs 216 of the anchor body 202 are engaged with the open slots 230 of the shaft 222 of the anchor driver 220. Each open slot 230 is configured to receive the respective rib 216 of the plurality of ribs.

Referring to FIG. 2, all of the prongs 232, 234 have inner surfaces 236. For each prong 232, 234, the inner surface 236 includes a protrusion 240. The protrusion 240 forms an interference fit with the respective rib 216 of the plurality of ribs 216 of the anchor body 202. The protrusion 240 can have any geometrical shape. In some embodiments, the protrusion 240 is a square. FIG. 2 illustrates an anchor driver 220 with four prongs, each prong have an inner surface 236 and each inner surface 236 includes a protrusion 240. The anchor driver 220 includes four open slots 230. Each open slot 230 is configured to receive the respective rib 216 of the plurality of ribs 216. Accordingly, the distal portion 228 of the anchor driver 220 has a square shape. However, the distal portion 228 can have any shape. For example, the distal portion 228 can have a square shape, D-shape, hexagonal shape, octagonal shape, or any geometrical shape.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Whereas many alterations and modifications of the disclosure will no doubt become apparent to a person of ordinary skill in the art after having read the foregoing description, it is to be understood that the particular embodiments shown and described by way of illustration are in no way intended to be considered limiting. Further, the subject matter has been described with reference to particular embodiments, but variations within the spirit and scope of the disclosure will occur to those skilled in the art. It is noted that the foregoing examples have been provided merely for the purpose of explanation and are in no way to be construed as limiting of the present disclosure.

Although the present disclosure has been described herein with reference to particular embodiments, the present disclosure is not intended to be limited to the particulars disclosed herein; rather, the present disclosure extends to all functionally equivalent structures, methods and uses, such as are within the scope of the claims.

1. A system for tissue repair comprising:
an anchor body having a proximal end, a distal end, and a plurality of turns of a screw thread extending between the proximal and distal ends, the plurality of turns of the screw thread defining an internal volume communicating with a region exterior to the anchor body through a spacing defined between the plurality of turns of the screw thread, and at least one rib disposed within the internal volume connected to at least two of the plurality of turns of the screw thread; and
an anchor driver comprising an elongated shaft extending between a proximal end and a distal end, a distal portion of the shaft including an open slot defined by at least two distally-extending prongs;
wherein the at least one rib of the anchor body is engageable with the open slot of the shaft of the anchor driver; and
wherein an inner surface of at least one of the at least two prongs defines a protrusion for forming an interference fit with the at least one rib.

2. The system for tissue repair of claim 1, wherein the anchor body comprises a plurality of ribs disposed within the internal volume.

3. The system for tissue repair of claim 2, wherein the anchor driver includes a plurality of open slots, each open slot is configured to receive the respective rib of the plurality of ribs.

4. The system for tissue repair of claim 1, wherein the inner surface of the at least one of the at least two prongs has a distal end and a proximal end.

5. The system for tissue repair of claim 4, wherein the protrusion is positioned on the distal end of the inner surface of the at least one of the at least two prongs.

6. The system for tissue repair of claim 4, wherein the protrusion is positioned on the proximal end of the inner surface of the at least one of the at least two prongs.

7. The system for tissue repair of claim 3, wherein each of the plurality of open slots is defined by two distally-extending prongs.

8. The system for tissue repair of claim 7, wherein an inner surface of at least one of the two prongs of each open slot defines a protrusion for forming an interference fit with the respective rib of the plurality of ribs.

9. A method of tissue repair comprising:
driving an anchor of an anchor system into a bone hole, the anchor system comprising:
an anchor body having a proximal end, a distal end, and a plurality of turns of a screw thread extending between the proximal and distal ends, the plurality of turns of the screw thread defining an internal volume communicating with a region exterior to the anchor body through a spacing defined between the plurality of turns of the screw thread, and at least one rib disposed within the internal volume connected to at least two of the plurality of turns of the screw thread; and
an anchor driver comprising an elongated shaft extending between a proximal end and a distal end, a distal portion of the shaft including an open slot defined by at least two distally-extending prongs;
wherein the at least one rib of the anchor body is engageable with the open slot of the shaft of the anchor driver, and wherein an inner surface of at least one of the at least two prongs defines a protrusion for forming an interference fit with the at least one rib; and
advancing the anchor body into the bone using the anchor driver.

10. The method of claim 9, wherein the anchor body comprises a plurality of ribs disposed within the internal volume.

11. The method of claim 10, wherein the anchor driver includes a plurality of open slots, each open slot is configured to receive the respective rib of the plurality of ribs.

12. The method of claim 9, wherein the inner surface of the at least one of the at least two prongs has a distal end and a proximal end.

13. The method of claim 12, wherein the protrusion is positioned on the distal end of the inner surface of the at least one of the at least two prongs.

14. The method of claim 12, wherein the protrusion is positioned on the proximal end of the inner surface of the at least one of the at least two prongs.

15. The method of claim 11, wherein each of the plurality of open slots is defined by two distally-extending prongs.

16. The method of claim 15, wherein an inner surface of at least one of the two prongs of each open slot defines a protrusion for forming an interference fit with the respective rib of the plurality of ribs.
